(19) United States
(12) Patent Application Publication
(10) Pub. No.: US 2008/0086053 A1
(43) Pub. Date: Apr. 10, 2008

(54) Component-Based Approach For Fast Left Ventricle Detection

(75) Inventors: Shaohua Kevin Zhou, Plainsboro, NJ (US); Jingdan Zhang, Plainsboro, NJ (US); Bogdan Georgescu, Plainsboro, NJ (US); Dorin Comaniciu, Princeton Junction, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS CORPORATE RESEARCH, INC., PRINCETON, NJ (US)

(21) Appl. No.: 11/866,712

(22) Filed: Oct. 3, 2007

Related U.S. Application Data
(60) Provisional application No. 60/849,935, filed on Oct. 6, 2006.

(51) Int. Cl. A61B 8/00

(52) U.S. Cl. 600/437

(57) ABSTRACT

A method for estimating a configuration of an internal structure within a medical image includes detecting a location of the internal structure. Component-based identification is performed within the detected location of the internal structure to identify a plurality of components. The configuration of the internal structure is estimated based on the relative position of the identified components.

### CROSS-REFERENCE TO RELATED APPLICATION

The present application is based on provisional application Ser. No. 60/849,935, filed Oct. 6, 2006, the entire contents of which are herein incorporated by reference.

### BACKGROUND OF THE INVENTION

1. Technical Field

The present disclosure relates to left ventricle detection and, more specifically, to a component-based approach for fast left ventricle detection.

2. Discussion of the Related Art

Echocardiography is the field of imaging the heart using an ultrasound. In echocardiography, all four chambers of the heart may be clearly viewed in a 2-dimensional image slice. An image sequence including multiple frames spanning a cardiac cycle may then be used to visualize cardiac function. Accordingly, certain cardiovascular disease may be diagnosed using data obtained from the echocardiograph. For example, the size and shape of the heart, pumping strength, and tissue damage may be observed. Moreover, the flow of blood through the heart may be observed to detect abnormal valve function, coronary artery disease and hypertrophic cardiomyopathy.

Computer-based detection may be used to provide automatic quantitative assessment of heart function based on echocardiographs. For example, the left ventricle may be identified and its characteristics may be accurately analyzed. The analysis of the left ventricle may then be used by a medical practitioner to diagnose and treat cardiac disease.

Computer-based detection may be performed either after the echocardiograph has been obtained, or in real-time. Real-time detection may provide additional utility as it may allow for instant diagnosis. Regardless of whether detection is performed in real-time or as a separate processing step, processing time is best kept to a minimum to increase efficiency and minimize costs associated with operating the image processing devices.

Many real-time object detection applications rely on a boosted cascade of Haar-like rectangle features. According to this approach, a complex distribution of training data may be learned efficiently by combining information obtained from a large number of relatively simple features that may be quickly and easily processed. Examples of this approach may be better understood with reference to B. Georgescu, S. Zhou, F. Comaniciu, A. Gupta: Database-Guided Segmentation of Anatomical Structures with Complex Appearance, IEEE Conf. Computer Vision and Pattern Recognition (CVPR'05), San Diego, Calif. 2005, which is herein incorporated by reference.

In performing real-time computer detection, it is often necessary to perform rigid transformation of the left ventricle image. Techniques for performing rigid transformation of the left ventricle may include an exhaustive search for the left ventricle within the echocardiograph. Because the orientation and scale of the left ventricle image may depend on the orientation of the patient and the manner in which the echocardiograph was taken, the search for the left ventricle must be able to adjust for differences in orientation and scale, for example, adjustment may occur within two translation parameters, one orientation parameter, and two scaling parameters.

Due to the intensity of the calculations necessary to find the left ventricle given this five-dimensional configuration space, and given the limitations of modern computational techniques and machinery, it may be very difficult to perform real-time computer detection. Accordingly, techniques have been developed to make these processing steps more efficient. For example, a coarse-to-fine approach may be implemented wherein the search process increases detail as the field of search narrows. However, even when using such approaches, detection speed may still be unacceptably slow.

For example, when performing computer-detection based on Haar-like features, estimation of the rotation parameter may be especially expensive in terms of computational resources. This is primarily because new integral images are calculated for each possible orientation. Moreover, when the size of the echocardiograph is large and the resolution is high, performing rotation for each possible orientation may be exceedingly difficult, computationally expensive, and time consuming. These factors limit the extent to which computer-detection may be performed in real-time and other such application requirements may not be met.

### SUMMARY

A method for estimating a configuration of an internal structure within a medical image includes detecting a location of the internal structure. Component-based identification is performed within the detected location of the internal structure to identify a plurality of components. The configuration of the internal structure is estimated based on the relative position of the identified components.

The medical image may be an echocardiograph. The internal structure may be a left ventricle of a heart. The plurality of components may include an apex or a valve annulus. The configuration of the internal structure may include the orientation of the internal structure. Performing component-based identification within the detected location of the internal structure to identify a plurality of components may include the use of rotation-invariant detectors, the use of detectors trained by Adaboost cascade techniques, and/or performing scale-invariant feature transforms (SIFT) or calculating histograms of oriented gradients.

The method may additionally include a training step for learning to discriminate the plurality of components from the medical image based on a set of training data. The training data may include rotation variations.

The method may additionally include estimating covariance matrices based on the identified plurality of components. Detection uncertainty may be modeled based on the estimated covariance matrices.

Performing component-based identification within the detected location of the internal structure to identify a plurality of components may include estimating a plurality of modes on a detection map. The center of each of the plurality of modes may then be found. A detection area corresponding to each of the-plurality of modes may be found. The detection area may be a partial fan area substantially originating from the center of the respective mode. A component of the plurality of components may be located within each of the detection areas.

A method for estimating a configuration of a left ventricle within an echocardiograph includes estimating a location of the left ventricle within the echocardiograph. A plurality of components are identified within the detected location of the left ventricle using rotation-invariant detectors. The configuration of the left ventricle is estimated based on the identified components.

The method may further include a training step for learning to discriminate the plurality of components from within the location of the left ventricle based on a set of training data. The training data may include rotation variations.

Covariance matrices may be estimated based on the identified plurality of components. Detection uncertainty may be modeled based on the estimated covariance matrices.

Performing component-based identification within the detected location of the internal structure to identify a plurality of components may include estimating a plurality of modes on a detection map. The center of each of the plurality of modes may be found. A detection area corresponding to each of the plurality of modes may be determined. The detection area may be a partial fan area substantially originating from the center of the respective mode. A component of the plurality of components may be located within each of the detection areas.

A computer system includes a processor and a program storage device readable by the computer system, embodying a program of instructions executable by the processor to perform method steps for estimating a configuration of a left ventricle within an echocardiograph. The method includes estimating a location of the left ventricle within the echocardiograph. A plurality of components are identified within the detected location of the left ventricle. The configuration of the left ventricle is estimated based on the identified components.

The method may additionally include a training step for learning to discriminate the plurality of components from within the location of the left ventricle based on a set of training data. Performing component-based identification within the detected location of the internal structure to identify a plurality of components may include estimating a plurality of modes on a detection map. The center of each of the plurality of modes may be found. A detection area corresponding to each of the plurality of modes may be determined. The detection area may be a partial fan area substantially originating from the center of the respective mode. A component of the plurality of components may be located within each of the detection areas.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 is a flow chart illustrating a method for estimating left ventricle configuration according to an exemplary embodiment of the present invention;

FIG. 2 is a flow chart detailing a possible implementation of the step of component based identification of FIG. 1 according to an exemplary embodiment of the present invention;

FIG. 3 is an echocardiograph showing training data according to an exemplary embodiment of the present invention;

FIGS. 4(A)-(F) show components and the corresponding search areas determined by the detection of the whole left ventricle according to an exemplary embodiment of the present invention; and

FIG. 5 shows an example of a computer system capable of implementing the method and apparatus according to exemplary embodiments of the present disclosure.

### DETAILED DESCRIPTION OF THE DRAWINGS

In describing the exemplary embodiments of the present disclosure illustrated in the drawings, specific terminology is employed for the sake of clarity. However, the present disclosure is not intended to be limited to the specific terminology so selected, and it is to be understood that each specific element includes all technical equivalents which operate in a similar manner.

Exemplary embodiments of the present invention seek to provide approaches to computer-detection of echocardiographs, for example, of the left ventricle, which may be performed more efficiently than known techniques. For example, exemplary embodiments of the present invention seek to efficiently estimate the configuration of the left ventricle while minimizing or eliminating the need to calculate new integral images for all possible orientations. Accordingly, computer-detection based on Haar-like features may be used to estimate translation and scaling parameters with greater efficiency and reduced computational expense.

Exemplary embodiments of the present invention may utilize a component-based detection approach for estimating the configuration of the left ventricle efficiently in echocardiograph images.

In an echocardiograph, there may be components such as the apex and valve annulus that present distinct structures that may be more easily identified. By efficiently identify all possible locations of these key components, a component-based detection approach can be applied to find the most likely configuration of components by matching the shape to acquired prior knowledge. Once the configuration of components are determined, the orientation of the left ventricle may be easily obtained.

Accordingly, component-based detection may be performed by detecting various possible locations of the key components. The key components may be detected using known techniques for detecting points of interest. Exemplary methods for detecting points of interest may utilize, scale-invariant feature transform (SIFT) features, Histogram of Oriented Gradient and the like. Herein, exemplary embodiments of the present invention may be described as using detectors trained by Adaboost cascade techniques to detect components. However, this approach is offered as an example of a technique that may be used, and exemplary embodiments of the present invention are not limited to this technique.

Adaboost cascade techniques may be able to capture the complex appearance of the components in a computationally efficient manner.

In order to minimize or avoid the use of computationally expensive image rotation, the trained detectors may be rotationally invariant, for example, the detectors searched for may be recognizable regardless of their rotational configuration.

During a training period, the boosting methods used herein may be able to directly learn how to discriminate the key components based on the training data. The training data may include examples with rotation variations. The classifiers trained in this way should be insensitive to rotation variations.

For rigid objects or near rigid objects, such as the left ventricle, the computational cost associated with detecting a component of an object may be even higher than detecting the whole object. This is because the appearance of the whole object may be more distinctive than that of its components. Accordingly, exemplary embodiments of the present invention may detect the location of the left ventricle, as a whole prior to performing component-based identification.

Component-based identification may then be used to estimate the orientation of the left ventricle. Accordingly the component detection may be performed in the areas constrained by the result of the whole left ventricle detection. In component detection, the key components may be identified, and based on the location of the various key components, the orientation of the left ventricle may be discovered.

Echocardiographs may include noise, signal drop-out and an overall complex appearance. These factors may contribute to uncertainty in component detection. Thus the detection map resulting from the component-based identification may include a level of uncertainty.

Covariance matrices may be estimated from the detection map. Known approaches may be used to estimate the covariance matrices, for example, as discussed in X. S. Zhou, D. Comaniciu, A. Gupta: An Information Fusion Framework for Robust Shape Tracking, IEEE Trans. Pattern Analysis Machine Intell., Vol. 27, No. 1, 115-129, 2005; and X. S. Zhou, D. Comaniciu, R. Cruceanu, B. Xie, A. Gupta: A Unified Framework for uncertainty Propagation in Automatic Shape Tracking, IEEE Conf. Computer Vision and Pattern Recognition (CVPR'04), Washington, D.C., 2004, both of which are herein incorporated by reference.

The estimated covariance matrices may then be used to model the detection uncertainty. This may be accomplished, for example, by using the estimated covariance matrices to perform an alignment process to determine a transformation function that transforms a mean shape for each of the key identified components to the detected shape of the identified component candidates. The mean shape for each of the key identified components may, for example, be determined from the training data. The determined transformation functions accordingly express the level of difference between the shape of the identified component candidates and their respective known shapes.

Accordingly, the orientation of the left ventricle may be estimated by first training a rotation-invariant classifier, based on a boosted cascade of simple features, to locate the possible position of the left ventricle. Then, the possible positions of each component of the left ventricle are detected in the limited area based on the located possible position of the left ventricle. Then, shape alignment may be applied, with uncertainty, to estimate the configuration of the left ventricle.

Accordingly, exemplary embodiments of the present invention may increase robustness and efficiency by limiting component detection to a region that has been identified as the location of the left ventricle, and then by using rotation-invariant detectors for component detection. In addition, missing components in shape alignment may be explicitly handled, as described below. Rotation-Invariant Classifiers for Left Ventricle Detection Rotation-invariant classifiers may be established in 2-dimentional echocardiographs by adding examples to the training data that include multiple different rotational configurations. The performance of the classifiers on detecting the left ventricle and components of left ventricle may be analyzed quantitatively.

Training data may include a set of echocardiograph images that each shows all four chambers of the heart. These images may be frames extracted from an annotated set of ultrasound heart image sequences. In one example, 260 such images may be used as training data. In this example, annotation includes a set of 17 control points of the left ventricle. A global transformation may be used to normalize each of the training images to a mean size. Here, the image size is 240×320.

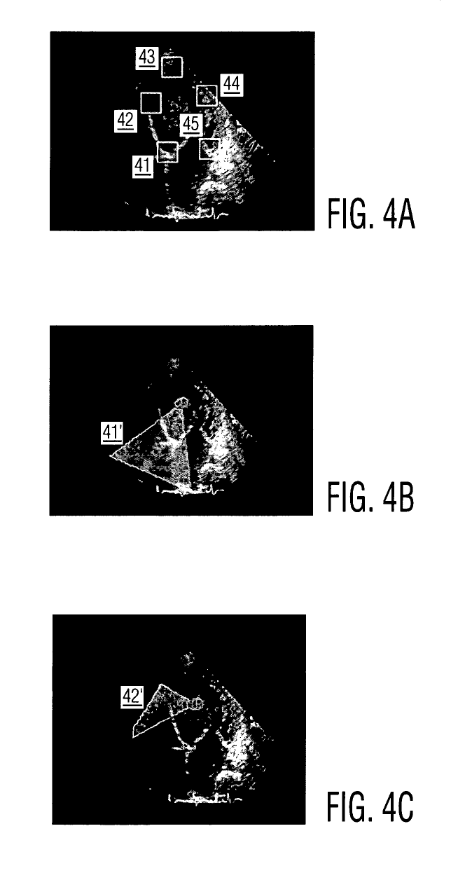

FIG. 3 is an echocardiograph showing training data according to an exemplary embodiment of the present invention. The regions of interest may be determined by the 17 control points 20, and these points may be used as positive samples. Accordingly, the region surrounded by the box 21 includes the positive samples 20. Negative samples may be taken from the regions whose center positions are outside the box 22.

The classifier may be trained using a boosted cascade of Haar-like features. There may be a maximum of 3 cascade levels. The maximum numbers of weak classifiers in each level are 10, 40, 200, respectively. The expected miss rate in all three levels is set to 0.

Training may be performed in two rounds. In the first round, negative data are randomly selected from the background areas. A classifier is trained and the false positive data of the training image are added to the negative set. Then the final classifier is trained using the updated negative set.

In order to build a training set including variations in rotational alignment, the normalized images may be rotated −25 and +25 degree respectively. Both rotated positive and negative data are added to the training set. Accordingly, the classifier may be made rotation-invariant.

Component Detection

Detecting the position of the whole left ventricle may be quicker and less computationally expensive than detecting an individual component of the left ventricle. Thus the position of the left ventricle may be detected first using the rotation-invariant detector. Then, the detected result may be used to constrain the search area within which the components are searched for. The geometric relation between the center of the left ventricle and components can be learned, for example, from the training data.

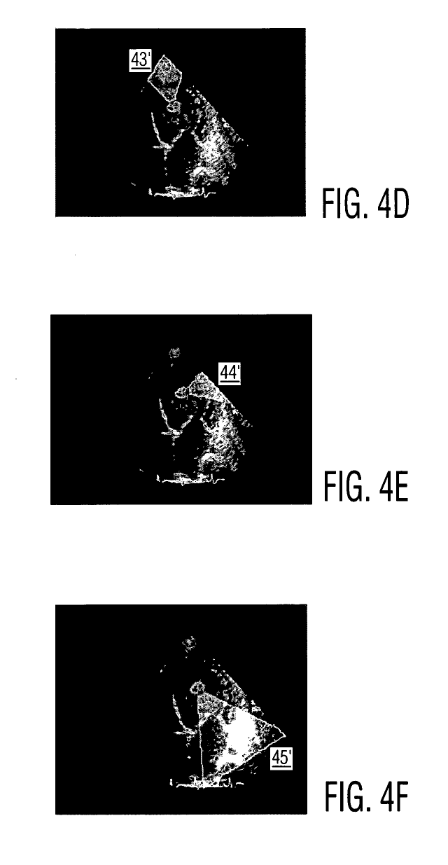

In performing the component detection within the established search area, first a set of modes may be estimated. The center of each of the estimated modes may be used to determine a detection area for each component. For example, a partial fan area may be determined according to the center of the each mode. FIGS. 4(A)-(F) show components and the corresponding search areas determined by the detection of the whole left ventricle.

In FIG. 4A, five components used in detection are shown 41-45. FIG. 4B shows the partial fan area 41′ corresponding to the component 41. Similarly, FIG. 4C shows the partial fan area 42′ corresponding to the component 42. FIG. 4D shows the partial fan area 43′ corresponding to the component 43. FIG. 4E shows the partial fan area 44′ corresponding to the component 44. FIG. 4F shows the partial fan area 45′ corresponding to the component 45. As can be seen from these figures, the center of each mode may be used to establish the partial fan-shaped detection areas 41′-45′ within which the respective components 41-45.

Shape Alignment

After each of the components has been identified, the identified components may be used to perform shape alignment which may be used to establish the configuration of the left ventricle. A detection map indicating the location of the detected regions may be established. The detection map may be noisy, for example, including heteroscedastic detection noise. Accordingly, in performing shape alignment modes may be found on detection map. The covariance matrices of the modes are estimated in the neighborhood of peak locations, for example, using a Hessian matrix, for example, in accordance with known techniques. Examples of such techniques may be found in Y. Kanazawa and K. Kanatani, Do we really have to consider covariance matrices for image features?, in Proc. Intl. Conf. on Computer Vision, Vancouver, Canada, volume II, 2001, pp. 586-591, which is herein incorporated by reference.

Multiple modes may be kept for each component to perform shape alignment.

The mean shape of each of the components may be obtained, for example, by applying Procrustes analysis to the training data. A set of pre-shapes can be determined by the combination of the candidate modes. In shape alignment, the most possible pre-shape and corresponding transformation between the mean shape and the pre-shape may be found by minimizing the following equation:

$${d}^{2} = {arg min}_{\{ T , {x}_{i} \}} {( {T}_{s} ( m ) - {x}_{i} )}^{T} {C}_{i}^{- 1} ( {T}_{s} ( m ) - {x}_{i} ) \qquad ( 1 )$$

where m is the mean shape, xᵢ is a pre-shape and Cᵢ is the corresponding covariance matrix. Tₛ is the similarity transform. To solve the equation (1), each possible pre-shape may be exhaustively searched. For each candidate pre-shape, the close-form solution may be obtained, for example using known techniques. Examples of such techniques may be found in T. Cootes and C. Taylor, Statistical models for appearance for computer vision, 2001 (unpublished manuscript available at http://www.wiau.man.ac.uk/bim/Models/app model.ps.gz.) which is hereby incorporated by reference.

Occlusion, however, may lead to the failure to detect one or more components. To handle this possibility, the pre-shape may be allowed to include a part of the component. In this situation, a penalty may be added to the equation (1).

In shape alignment, the left ventricle location found in the initial step may also be considered a component. Accordingly, the modes estimated in the first stage detection may be used to jointly estimate Tₛ. By estimating Tₛ, the orientation, position and scale of the left ventricle may be obtained.

Estimation of Left Ventricle Configuration

As discussed above, exemplary embodiments of the present invention are explained in terms of providing an estimation for the configuration of the left ventricle. However, the invention is not so limited, and the techniques discussed herein may be used to estimate the configuration of other structures. For example, these techniques may be used to estimate the configuration of a right ventricle. Moreover, exemplary embodiments of the present invention may be performed for 3-dimentional echocardiographs and/or medical images other than echocardiographs, for example, MRIs and CT scans. In such cases, these techniques may be used to estimate the configuration of internal structures not related to the heart.

However, for the purposes of presenting a clear description of the exemplary embodiments of the present invention, examples illustrated herein focus on estimation of left ventricle configuration. Left ventricle configuration, as used herein, includes the shape, position and orientation of the left ventricle.

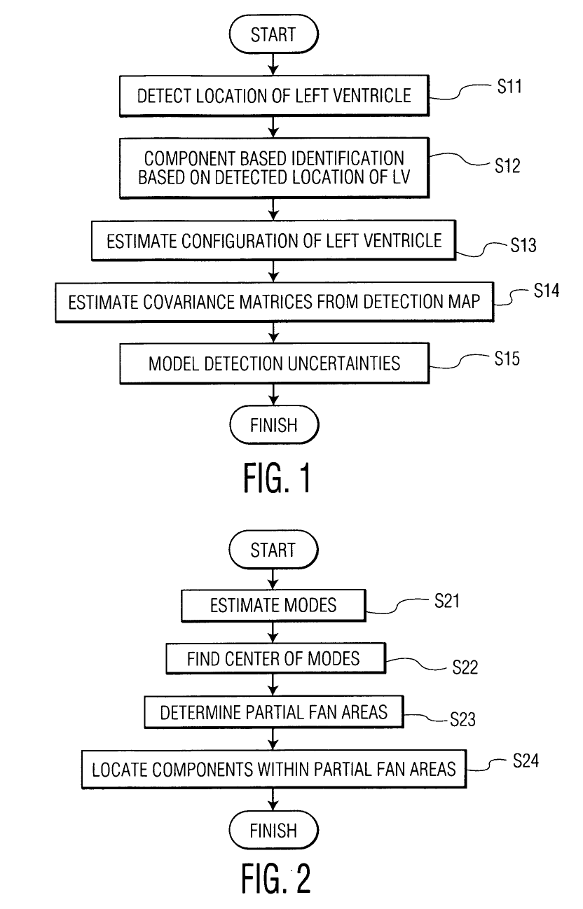

FIG. 1 is a flow chart illustrating a method for estimating left ventricle configuration according to an exemplary embodiment of the present invention. First, the location of the left ventricle may be detected (Step S11). In this step, the location of the left ventricle may be detected as a whole and prior to performing component-based identification.

Next, component-based identification of key components may be performed within the location of the left ventricle detected in the previous step (Step S12). Key components may be any components that may be relatively easy to identify and would help to determine the configuration of the left ventricle. For example, key components may include an apex and a valve annulus. The locations of the detected key components over the echocardiograph may comprise a detection map.

The configuration of the left ventricle may then be estimated based on the location of the detected key components found within the location of the left ventricle detected in step S11 (Step S13).

Finally, where it is desired that the level of detection uncertainty be known, covariance matrices may be estimated from the detection map (Step S14) and the estimated covariance matrices may then be used to model the detection uncertainty (Step S15).

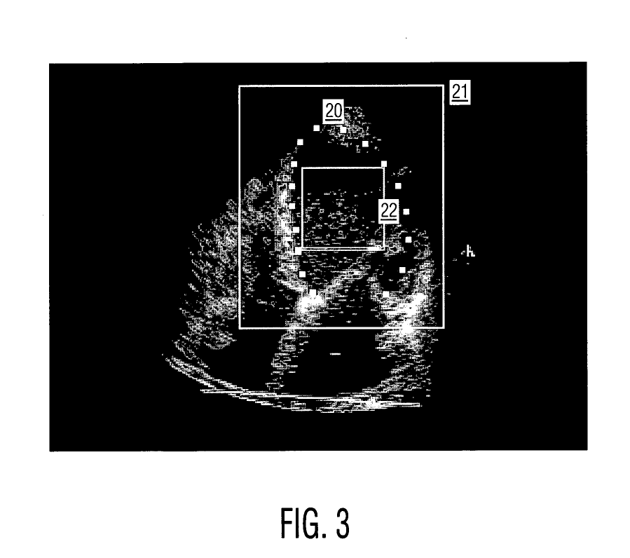

FIG. 2 is a flow chart illustrating the step of component based identification (Step S12) described above with respect to FIG. 1. With respect to FIG. 2, first, a set of modes may be estimated on the detection map of the left ventricle (Step S21). Then, the center of each mode may be found (Step S22). The center of each mode may be used to determine a detection area for each component (Step S23). Each detection area may be a partial fan area on the detection map. Then, each component may be located within a respective detection area (Step S24).

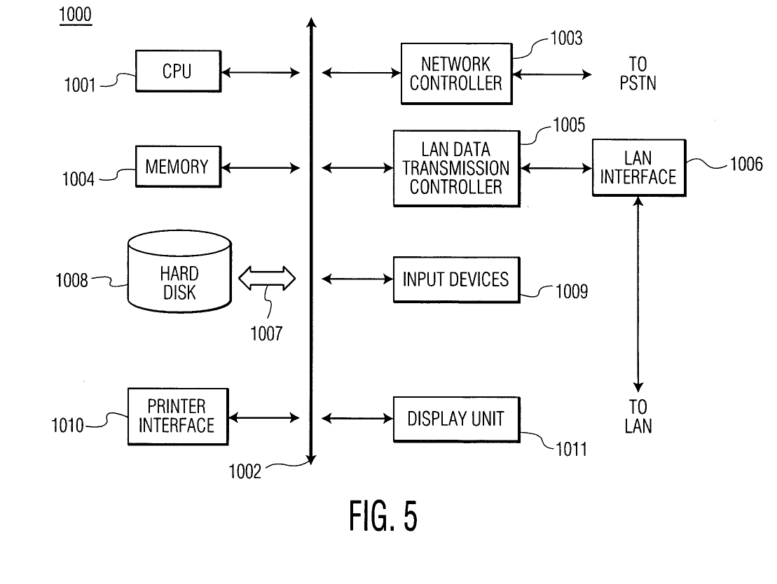

FIG. 5 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007.

The above specific exemplary embodiments are illustrative, and many variations can be introduced on these embodiments without departing from the spirit of the disclosure or from the scope of the appended claims. For example, elements and/or features of different exemplary embodiments may be combined with each other and/or substituted for each other within the scope of this disclosure and appended claims.

What is claimed is:

1. A method for estimating a configuration of an internal structure within a medical image, comprising:
detecting a location of the internal structure;
performing component-based identification within the detected location of the internal structure to identify a plurality of components; and
estimating the configuration of the internal structure based on the relative position of the identified components.

2. The method of claim 1, wherein the medical image is an echocardiograph.

3. The method of claim 1, wherein the internal structure is a left ventricle of a heart.

4. The method of claim 1, wherein the plurality of components include an apex or a valve annulus.

5. The method of claim 1, wherein the configuration of the internal structure includes the orientation of the Internal structure.

6. The method of claim 1, wherein performing component-based identification within the detected location of the internal structure to identify a plurality of components includes the use of rotation-invariant detectors.

7. The method of claim 1, wherein performing component-based identification within the detected location of the internal structure to identify a plurality of components includes the use of detectors trained by Adaboost cascade technique.

8. The method of claim 1, wherein performing component-based identification within the detected location of the internal structure to identify a plurality of components includes performing scale-invariant feature transforms (STFT) or calculating histograms of oriented gradients.

9. The method of claim 1, additionally including a training step for learning to discriminate the plurality of components from the medical image based on a set of training data.

10. The method of claim 10, wherein the training data includes rotation variations.

11. The method of claim 1, additionally comprising:
estimating covariance matrices based on the identified plurality of components; and
modeling detection uncertainty based on the estimated covariance matrices.

12. The method of claim 1, wherein performing component-based identification within the detected location of the internal structure to identify a plurality of components includes:
estimating a plurality of modes on a detection map;
finding the center of each of the plurality of modes;
determining a detection area corresponding to each of the plurality of modes, wherein the detection area is a partial fan area substantially originating from the center of the respective mode; and
locating a component of the plurality of components within each of the detection areas.

13. A method for estimating a configuration of a left ventricle within an echocardiograph, comprising:
estimating a location of the left ventricle within the echocardiograph;
identifying a plurality of components within the detected location of the left ventricle using rotation-invariant detectors; and
estimating the configuration of the left ventricle based on the identified components.

14. The method of claim 13, additionally comprising a training step for learning to discriminate the plurality of components from within the location of the left ventricle based on a set of training data.

15. The method of claim 14, wherein the training data includes rotation variations.

16. The method of claim 13, additionally comprising:
estimating covariance matrices based on the identified plurality of components; and
modeling detection uncertainty based on the estimated covariance matrices.

17. The method of claim 13, wherein performing component-based identification within the detected location of the internal structure to identify a plurality of components includes:
estimating a plurality of modes on a detection map;
finding the center of each of the plurality of modes;
determining a detection area corresponding to each of the plurality of modes, wherein the detection area is a partial fan area substantially originating from the center of the respective mode; and
locating a component of the plurality of components within each of the detection areas.

18. A computer system comprising:
a processor; and
a program storage device readable by the computer system, embodying a program of instructions executable by the processor to perform method steps for estimating a configuration of a left ventricle within an echocardiograph, the method comprising:
estimating a location of the left ventricle within the echocardiograph;
identifying a plurality of components within the detected location of the left ventricle; and
estimating the configuration of the left ventricle based on the identified components.

19. The computer system of claim 17, wherein the method additionally comprises a training step for learning to discriminate the plurality of components from within the location of the left ventricle based on a set of training data.

20. The computer system of claim 17, wherein performing component-based identification within the detected location of the internal structure to identify a plurality of components includes:
estimating a plurality of modes on a detection map;
finding the center of each of the plurality of modes;
determining a detection area corresponding to each of the plurality of modes, wherein the detection area is a partial fan area substantially originating from the center of the respective mode; and
locating a component of the plurality of components within each of the detection areas.
